(19) United States
(12) Patent Application Publication
(10) Pub. No.: US 2007/0150000 A1
(43) Pub. Date: Jun. 28, 2007

(54) DEVICE FOR CLOSING AN OPENING FORMED BY TWO OVERLAPPING TISSUE FLAPS IN THE HEART

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/613,299

(22) Filed: Dec. 20, 2006

(30) Foreign Application Priority Data
Dec. 28, 2005 (DE) 102005062658.0-35

(51) Int. Cl. A61B 17/08

(52) U.S. Cl. 606/218

(57) ABSTRACT

A device (1) for closing an opening (2) (PFO-closure) formed by two overlapping tissue flaps or skin folds (5) and (6) in the heart (3) is provided, and includes a feeding catheter (7), that can be inserted into the heart (3), transvenously for the transvenous introduction of a device that is to connect the two tissue flaps (5) and (6) to each other. The device includes a threaded catheter (8) that can be pushed forward inside the feeding catheter (7) and can be pushed out at a distal end thereof, with a thread (9) arranged at the threaded catheter distal end to be screwed into the tissue flap (5) positioned closer to the feeding catheter (7) in the operational condition. A tension catheter (10) can be connected through the tissue flap closer to the guidance catheter (7) after its puncture, to the tissue flap (6) positioned therebehind. The tension catheter (10) can be a second threaded catheter with a thread (11) or a suction pipe. In this manner, the rear tissue flap (6) can be pulled towards the first tissue flap (5) until they contact each other. A high-frequency catheter contacting the first tissue flap (5) can be provided with a pole and the tension catheter (10) serving to pull the second tissue flap (6) closer can serve as the counter electrode with another pole, and between the two electrodes or poles a high frequency voltage can be provided and the two electrodes are spaced at a distance from each other in an operational state and/or are isolated from each other in order to allow a coagulation of the contact site of the two tissue flaps (5) and (6).

### BACKGROUND

The invention relates to a device for closing an opening formed by two overlapping tissue flaps in the heart (PFO-closure) in a separating wall located in the heart between two ventricles, particularly between the right atrium and the left atrium, having a feeding catheter that can be inserted into the heart transvenously for the transvenous introduction of a device for connecting the tissue flaps to each other, having a tension catheter that can connect the closer tissue flap with the tissue flap located therebehind by the feeding catheter penetrating the closer tissue flap, with said feeding catheter and the rear tissue flap being pulled against the frontal tissue flap until mutual contact is established, using a high frequency catheter having a pole and contacting the first tissue flap, and the tension catheter serving to pull the second tissue flap being embodied as a counter electrode having another pole, such that a high frequency voltage can be applied between the two electrodes or poles with the two electrodes being distanced from each other and/or isolated from each other.

Such a device is known from U.S. 2005/0192654 A1 and is here primarily shown in the FIGS. 10A through 10E and FIGS. 14 through 16. In order to allow the tension catheter to engage the rear tissue flap in the direction of feed, it must be moved together with an anchor radially protruding therefrom through both tissue flaps, requiring a second feeding catheter inside the first feeding catheter, which simultaneously carries a stop in the feeding direction for the frontal or first tissue flap. This second interior feeding catheter for the tension catheter must be embodied at its end, initially accepting the tension catheter with the anchor, as a hollow needle with a sharp rim, in order to allow the penetration of the two tissue flaps.

However, even with sharply cut faces of this hollow-needle type end there is the risk that the edges of the opening slip inside the separating wall and/or the adjacent tissue flaps due to its low rigidity so that the cutting rim of the hollow needle and/or the hollow-needle type second feeding catheter do not penetrate the tissue flaps and thus the tension catheter does not reach the operating position, thus preventing the placement of the anchor element.

### SUMMARY

Therefore, the object is to provide a device of the type mentioned at the outset, by which the opening located in a separating wall in the heart between overlapping tissue flaps or skin folds can be closed by way of a transvenous feeding, with the slipping of the tissue flap or flaps being avoided when the device impinges.

In order to attain this objective, the device defined at the outset with an arrangement for connecting the tissue flaps is characterized in that the connection device according to the invention has a threaded catheter to be pushed out at its distal end provided with a thread that can be screwed into the tissue flap closer to the feeding catheter in the operating position and that the tension catheter for pulling the second tissue flap towards the first tissue flap is provided as a threaded catheter with at least one thread, which can be pushed through the first threaded catheter and with its thread or threads being insertable into the second tissue flap facing away from the feeding catheter, or that a suction pipe or hose is provided as a tension catheter, which can be pushed through the screwed-in catheter and its thread and a functional opening in the first tissue flap facing the feeding catheter to the second tissue flap overlapping it, and with its proximal end being connectable to a vacuum source, and that said suction pipe can additionally be connected to a high frequency voltage source or a high frequency generator.

Therefore, using the threaded catheter the tissue flap located closer to the feeding catheter of the opening to be closed can be prevented from slippage during further operations, such that it is engaged by a thread or if necessary by two or more threads of the threaded catheter, staggered in the circumferential direction, equally sized, aligned with each other, and having an identical incline, so that, when a pressure is applied, an appropriate counter tension can be applied by this threaded catheter. This way, the first tissue flap can be penetrated without slippage and thus the second tissue flap can be reached, which in turn can be pulled towards the first tissue flap by the tension catheter, and subsequently the two tissue flaps can be permanently connected by a local coagulation via high frequency currency, if necessary at several places and then grow together. Due to the fact that the catheter and the electrodes can be removed no “foreign objects” remain in the body or the heart in this PFO-closure.

Due to the fact that the tension catheter serving to pull the second tissue flap towards the first tissue flap can also be embodied as a threaded catheter having at least one thread which can be pushed through the first threaded catheter and with its thread or threads can be screwed into the second tissue flap facing away from the feeding catheter, from the second threaded catheter, which again can be fed transvenously through the first threaded catheter and thus also through the feeding catheter, a mechanical connection can be made to the second tissue flap so that it can easily brought into contact by pulling the second threaded catheter into the contact position with the first tissue flap without risking any slippage of one of the tissue flaps.

Alternatively, a suction pipe or hose can be provided as the tension catheter, which comprises electrically conducting material or is provided with an electric conductor such that it can be connected to the high-frequency generator and can form the second electrode with the second pole for the high-frequency coagulation.

Here, it is advantageous when the first threaded catheter is embodied as a high-frequency catheter and can be connected to a high-frequency generator. Furthermore, it is here particularly advantageous for the second threaded catheter to be embodied as a counter electrode, which can be connected to a high-frequency generator. In such an embodiment, the two threaded catheters form the electrodes or poles for applying the high-frequency current for a permanent connection of the two tissue flaps, before they finally and completely grow together.

Due to the fact that the parts of the device can be inserted transvenously the surgical expense for this PFO-closure is relatively low.

This device may be provided with a puncture instrument and/or a dilator, with the instrument being able to advance through the feeding catheter and/or through the first threaded catheter and which, for the formation of a penetrating opening in the interior area of the first thread, can also be pierced through the first tissue flap. With the help of a puncture instrument or dilator, after the thread of the first threaded catheter has been screwed in, an opening can be made in the first tissue flap through the area of the first tissue flap enclosed by said thread, which facilitates the feeding of the tension catheter, for example a second threaded catheter, to the rear tissue flap in the feeding direction.

Through individual or a combination of several of the above-described features and measures, a PFO-closure results by heating using high-frequency current. Here, the tissue flap positioned closer to the transvenously inserted feeding catheter and/or a skin fold in the separating wall of the heart can be engaged and fixed by the thread of the threaded catheter brought into the operational position through the feeding catheter. Subsequently, the frontal tissue flap, closer to the feeding catheter, can be punctured, and then a tension catheter, also embodied as a threaded catheter with a small diameter or as a suction pipe or hose can be brought into the area of the rear tissue flap through the skin fold and/or the first tissue flap punctured and held with the help of the first threaded catheter, and here it is connected by screwing in or by a vacuum such that said rear tissue flap can be pulled to the front one by pulling the tension catheter. Here, with the help of high frequency voltage using a conventional high-frequency generator, a local heating of the two tissue flaps or skin folds can occur, followed by a mechanical connection and/or a adhering of the two skin folds to each other. This results in the two tissue flaps or skin folds growing together to completely close the PFO-opening in this manner. The local coagulation via high-frequency current may also be performed at several places here, depending on the size of the opening.

Here, it is also possible for several feeding catheters to be provided at a high-frequency catheter and a counter electrode, which can be connected to a high-frequency generator, or for the feeding catheter being used with a threaded catheter and the high-frequency catheter as well as the mating electrode at several neighboring places.

### BRIEF DESCRIPTION OF THE DRAWINGS

In the following, exemplary embodiments of the invention are described in greater detail using the drawing. In a partially schematic representation, shown are:

FIG. 1 is a side view of the device according to the invention having a feeding catheter and a threaded catheter arranged therein and displaceable through it, with its proximal end protruding at the rear of the guidance catheter and provided with a holding grip for shifting and rotating and manipulating, while at the distal end of said threaded catheter, a thread is provided, which protrudes from the feeding catheter in the position shown;

FIG. 2 is a cross-sectional view through a heart and through the left and right atrium, with an opening existing in the separating wall between said two atria, which is limited by two overlapping tissue flaps or skin folds of said separating wall and with the guidance catheter with the threaded catheter being inserted transvenously into the right atrium and with its mouth approaching the separating wall such that the thread of the threaded catheter can be screwed into the tissue flap closer to the guidance catheter and/or in the graphic representation has already been screwed in, and by the threaded catheter hinders this tissue flap from slippage when another instrument engages, in particular a puncture instrument and/or dilator;

FIG. 3 is an enlarged scale view of the distal ends of the feeding catheter and of the threaded catheter protruding therefrom in the situation according to FIG. 2, showing the distal end of the feeding catheter and the threaded catheter advancing therein after the thread was screwed into the tissue flap of the separating wall of the heart closer to the feeding catheter;

FIG. 4 is a representation according to FIG. 3 after the advance of the puncture instrument through the center of the thread of the first threaded catheter and through the first tissue flap, with said flap being held back against the force developing during the penetration of the puncture instrument by the thread and the threaded catheter;

FIG. 5 is a representation according to FIGS. 3 and 4 after the puncture and dilation as well as after the insertion of the second threaded catheter through the first threaded catheter and through the tissue flip located closer to the guiding catheter and after its thread has been screwed into the tissue flap located in the rear in the penetration direction, with an insulation being provided at said second threaded catheter so that the two threaded catheters can serve as electrodes that can be connected to a high-frequency generator as different high-frequency poles;

FIG. 6 is a representation according to FIG. 5 after the second tissue flap has been pulled into a contacting position in reference to the first tissue flap with the help of the second threaded electrode, after which, by a local coagulation, both tissue flaps can be connected to each other in this position so that they are then growing together;

FIG. 7 is a representation according to FIG. 5, in which, after the mounting of the first threaded electrode and the puncture of the tissue flap engaged thereby as a tension catheter, the second electrode is not provided with a second thread but embodied as a suction pipe or suction hose advanced towards the second or rear tissue flap and engaging it via a vacuum provided at said suction pipe; and

FIG. 8 is a representation according to FIG. 6 after the rear tissue flap, as seen by the guidance catheter, has been pulled to the frontal tissue flap by a relative tension being applied to the suction pipe, after which the two tissue flaps are adhered together via high-frequency.

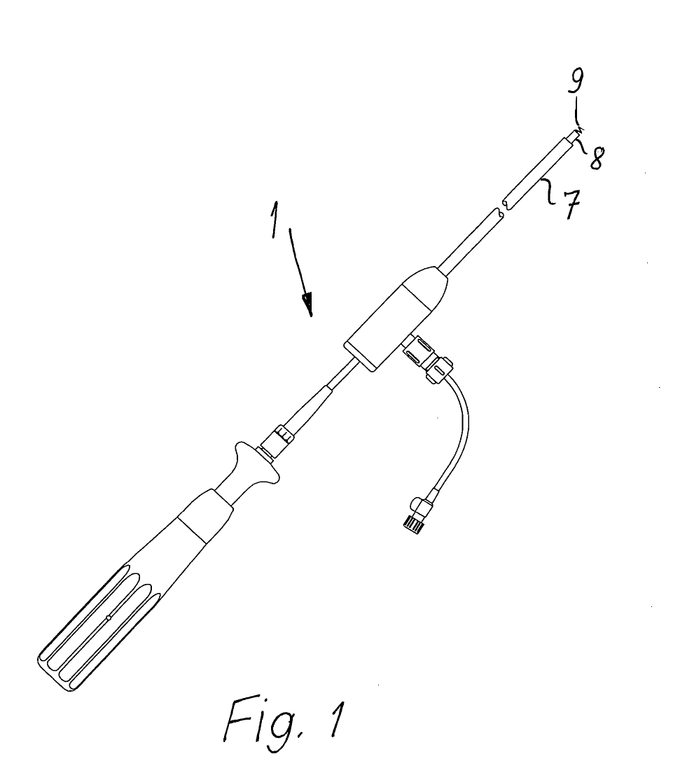

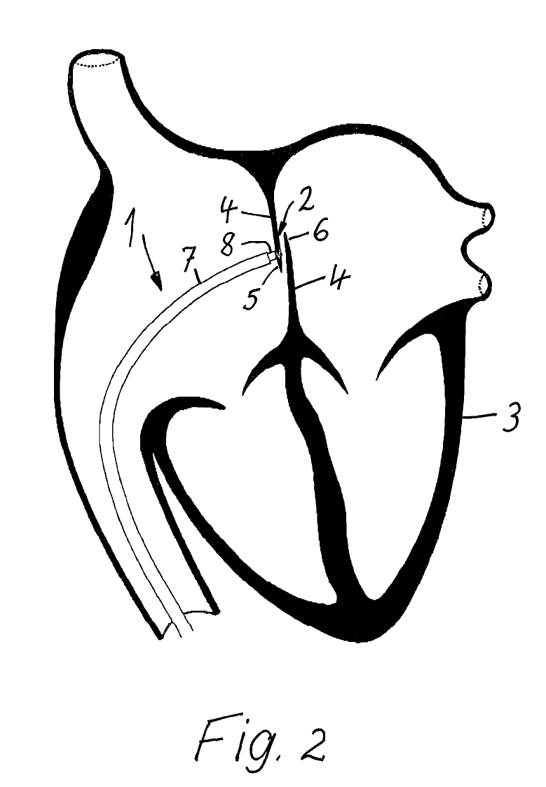

### DETAILED DESCRIPTION OF THE PREFERRED EMBODIMENTS

A device, indicated as 1 in its entirety, also called or seen as a construction set, serves to close an opening 2 in a separating wall 4 located in the heart 3 between two ventricles, in particular between the right and the left atrium, with said opening 2 being limited by two overlapping tissue flaps 5 and 6.

Here, the parts and objects identical in their function have identical reference numerals even if the embodiments are different.

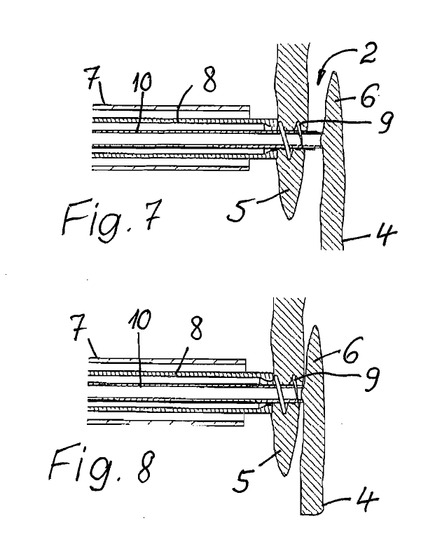

The device 1 serves to create a PFO-closure and has a feeding catheter to be inserted into the heart 3 transvenously for the transvenous insertion of a device to be described in the following for connecting the tissue flaps 5 and 6 to each other. This device for connecting the two tissue flaps 5 and 6 is provided with a threaded catheter 8, that can be pushed through the feeding catheter 7 and with its distal end exits at the distal end thereof, and has at its distal end a thread 9, which can be screwed into the tissue flap 5 located closer to the feeding catheter 7 in the operational position, and additionally a tension catheter 10, which can connect the tissue flap 6 located in the rear with the closer tissue flap 5 through the guidance catheter 7. The tension catheter 10 may be embodied in different manners remaining to be described, according to FIGS. 5 and 6 on the one hand and according to FIGS. 7 and 8 on the other hand. Here, several, preferably two identical threads 9, off-set in reference to each other, are provided, which can engage the tissue in an even more effective manner.

With the help of this tension catheter 10 the rear tissue flap 6, located at the side facing away from the guidance catheter 7, according to FIGS. 6 and 8, can be pulled to the first tissue flap 5 until they contact each other.

For the mutual connection of the two tissue flaps 5 and 6 it is provided that one high-frequency catheter contacting the first tissue flap 5, namely the first threaded catheter 8, that can be connected to a high-frequency generator, is embodied as the first pole, for example its thread, and the tensile catheter 10, serving to pull the second tissue flap 6, is embodied as a counter electrode with another pole. Between these two electrodes or poles, in a manner not shown, a high-frequency voltage can be created at the proximal end of the device 1, in order to allow, with the help of a high-frequency current, the now contacting tissue flaps 5 and 6 to coagulate and be adhered together locally in order to initiate their growing together.

The two electrodes and poles are here at a distance and/or isolated from each other in the operational state and/or insulated in order to avoid short-circuiting.

As already mentioned, here the first threaded catheter 8 is embodied with a thread 9 as a high-frequency catheter and can be or will be connected to a high-frequency generator.

The tension catheter 10, serving to pull the second tissue flap 6 towards the first tissue flap 5, is embodied in the exemplary embodiment according to FIGS. 5 and 6 as a second threaded catheter having a thread 11, which can be pushed through the first threaded catheter 8, and its thread 9 and which can be screwed into the second tissue flap 6 positioned away from the feeding catheter 7, as shown in FIG. 5. In this case, the second threaded catheter with the thread 11 is embodied as a counter electrode and can be connected to a high-frequency generator so that two electrodes and poles isolated from each other are available for the desired coagulation. Here, again, two or more threads 11 identical to each other can be provided.

This device 1 includes in both exemplary embodiments a puncture instrument 12, with its distal end being embodied as a dilator and being displaceable through the feeding catheter 7 and the first threaded catheter 8 and its thread 9, and serving to form a penetrating opening in the interior area of said first thread 9 in the first tissue flap 5. The effectiveness of the functional instrument 12 and its tapering located at its end are discernible in FIG. 4. This way, the introduction and the screwing in of the second threaded catheter with its thread 11 are facilitated, while during the puncture and also during the insertion of the second thread 11 the first tissue flap 5 is prevented from slippage by the first threaded catheter 8 and its thread 9 such that an appropriate tensile force is acted upon said first threaded catheter 8.

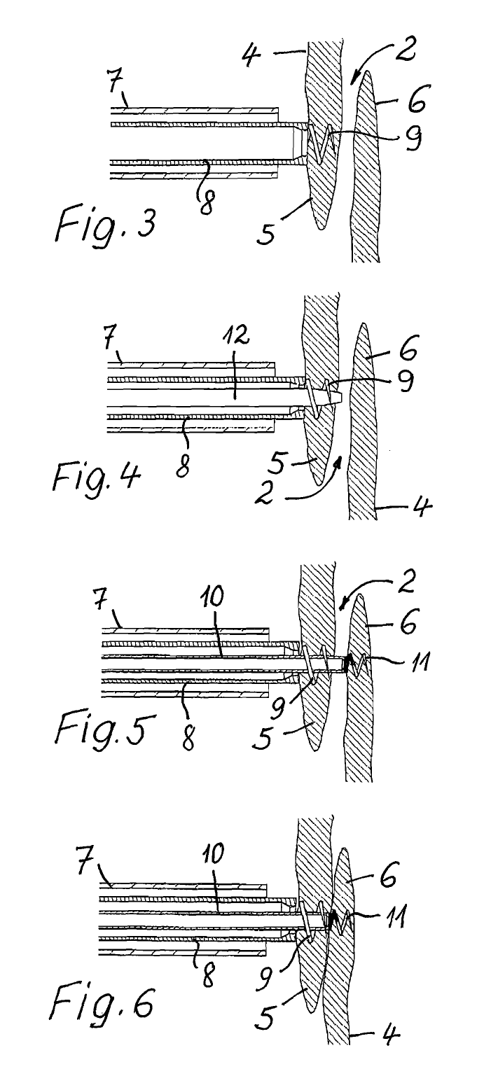

A modified embodiment is seen in FIGS. 7 and 8, with for this embodiment FIGS. 3 and 4 also applying, because this second embodiment, also includes a first threaded catheter 8 with a thread 9 and a functional element 12 with a dilator.

Here, a suction pipe or hose is provided as a tension catheter 10, which can be displaced after the puncture through the threaded catheter 8 and its thread 9 as well as through the puncture opening in the first tissue flap 5 facing the feeding catheter 7 to the second tissue flap 6 overlapping said first tissue flap 5, and can be connected with its proximal end to a vacuum source, in a manner not shown in greater detail here, so that, based on this vacuum, through a tension force to said suction hose the rear tissue flap 6 is pulled into contact with the frontal tissue flap 5 as shown in FIG. 8. Here, the suction pipe, serving as the tension catheter 10, is again embodied as a counter electrode and can be connected to a high-frequency generator, so that in this exemplary embodiment a high-frequency current can flow between the tension catheter 10 and the threaded catheter 8 for coagulating and connecting the two tissue flaps 5 and 6.

This mutual connection of the two tissue flaps 5 and 6 according to FIGS. 6 and 8 can here be performed, depending on the size of the opening 2, several times next to each other. A device 1 having several feeding catheters 7 may also be provided with a high-frequency catheter and a counter electrode that can be connected to a high-frequency generator.

In the two exemplary embodiments of a device 1 for a PFO-closure via heating by high-frequency current first the frontal tissue flap 5, positioned closer to the feeding catheter 7, is fixed with the help of a first threaded catheter 8 and a thread 9. Then this tissue flap 5 is puncture, i.e. pierced, in the center of the penetrating thread 9 so that a tension catheter flap 6 located therebehind, and the two catheters then can be connected to a high-frequency generator such that between them, with appropriate isolation, a high-frequency current flows through both tissue flaps 5 and 6 and they “adhere together” by coagulation when they mutually touch. This mutual contact can be provided with the help of a second thread 11 of a second threaded catheter or with the help of a suction pipe and a tensile force applied here.

A device 1 for closing an opening 2 (PFO-closure) formed in the heart 3 using two overlapping tissue flaps or skin folds 5 and 6 is provided with a feeding catheter 7 that can be inserted transvenously into the heart 3 for the transvenous introduction of a device, by which the two overlapping tissue flaps 5 and 6 are to be connected together. This device essentially comprises a threaded catheter 8 that can be displaced in the feeding catheter 7 and can be pushed out of its distal end, with a thread 9 arranged at the distal end, that can be screwed into the tissue flap 5 located closer to the feeding catheter 7 in the operational state, and having a tension catheter 10, with the tension catheter 10 that can be connected through the tissue flap positioned closer to the guiding catheter 7 after its puncture with the tissue flap 6 located therebehind, the tension catheter may be a second threaded catheter with a thread 11 or a suction pipe. Using the tension catheter, the rear tissue flap 6 can be pulled to the first tissue flap 5 until they contact each other. The catheter contacting the first tissue flap 5 can be provided with a pole supplied with high frequency energy, and the tension catheter 10, serving to pull the second tissue flap 6, may be embodied as a counter electrode with another pole, and a high-frequency voltage may be connected between said two electrodes or poles and the two electrodes are at a distance from each other and/or isolated from each other in order to allow a coagulation of the contact site of the two tissue flaps 5 and 6.

It must be mentioned that the first threaded catheter 8 with its thread 9, after having pulled the second tissue flap 6 towards the first tissue flap 5, can be removed and replaced by an independent high-frequency catheter, which then cooperates with the pole located at the tension catheter 10 in the same manner and can cause a coagulation. In such an embodiment, not shown, the first threaded catheter 8 is not required to be electrically conducting and/or insulated and to be provided with a connection device for a high-frequency generator. Rather, a high-frequency generator can be subsequently pushed forward through the feeding catheter 7, which guides it until it touches the first tissue flap 5.

1. A device (1) for closing an opening (2) formed by two overlapping tissue flaps (5, 6) in a separating wall located in a heart (3) between two ventricles, between the right atrium and the left atrium, the device comprising a feeding catheter (7) that can be introduced intravenously into the heart (3) for intravenous introduction of the device for connecting the tissue flaps (5, 6) with each other, a tension catheter (10) that can be connected through the tissue flap (5), located closer to the feeding catheter (7), to the tissue flap (6) located therebehind, the tension catheter (7) being adapted to allow the rear tissue flap (6) facing away from the feeding catheter (7) to be pulled against the first tissue flap (5) for a mutual contact, a high-frequency catheter contacting the first tissue flap (5) having a first pole, and the tension catheter (10) which serves to pull the second tissue flap (6) closer having a pole embodied as a counter electrode to act with the first pole so that between said two electrodes or poles a high-frequency voltage can be applied and the two electrodes are spaced apart from each other and/or isolated from each other, the device for connecting comprising a threaded catheter (8), that can be displaced inside the feeding catheter (7) and can be pushed out at a distal end thereof, including at least one thread (9) that can be screwed into the tissue flap (5) closer to the feeding catheter (7) in an operating position, arranged at a threaded catheter distal end, and the tension catheter (6) for pulling the second tissue flap (6) close to the first tissue flap (5) comprises a second threaded catheter with at least one thread (11), which can be pushed through the first threaded catheter (8) and with a thread or threads thereof can be screwed into the second tissue flap (6) positioned away from the feeding catheter (7), or the tension catheter (6) comprises a suction pipe or hose which can be pushed through the threaded catheter (8) and the thread (9) thereof and a puncture opening in the first tissue flap (5) facing the feeding catheter (7) to the second tissue flap (8) overlapping it and can be connected with a proximal end thereof to a vacuum source, and said suction pipe can additionally be connected to a high-frequency generator as the counter electrode.

2. A device according to claim 1, wherein the first threaded catheter (8) comprises a high-frequency catheter and can be connected to a high-frequency generator.

3. A device according to claim 1, wherein the second threaded catheter comprises the counter electrode and can be connected to a high-frequency generator.

4. A device according to claim 1, further comprising a puncture instrument (12) and/or a dilator, and the puncture instrument is adapted to be pushed through the feeding catheter (7) and/or through the first threaded catheter and which, in order to form a penetrating opening in an interior area of the first thread (9), can pierce through the first tissue flap (5).
